(19) United States
(12) Patent Application Publication
(10) Pub. No.: US 2011/0275076 A1
(43) Pub. Date: Nov. 10, 2011

(54) BULKED MUTANT ANALYSIS (BMA)

(75) Inventor: Jeroen Stuurman, Oosterbeek (NL)

(73) Assignee: KEYGENE N.V.

(21) Appl. No.: 13/129,512

(22) PCT Filed: Nov. 17, 2009
(86) PCT No.: PCT/NL09/00222

§ 371 Date: Jul. 26, 2011

Related U.S. Application Data
(60) Provisional application No. 61/115,239, filed on Nov. 17, 2008.

(51) Int. Cl. C12Q 1/68

(52) U.S. Cl. 435/6.11

(57) ABSTRACT

The current invention relates to a new strategy for identification, and optional isolation, of a nucleic acid sequence that is expressed in an organism and that is related to a particular phenotype (trait of a character) of said organism. With the method of the current invention it has become possible to, in contrast to known methods in the art, efficiently identify, isolate or clone genes in, for example, organism like (crop) plants for which no or only limited information with respect to the genome is available.

### BACKGROUND TO THE INVENTION

Cloning genes from mutant phenotypes has been a longstanding challenge in genetics and biotechnology. In particular in the field of (crop) plants, rapid and cheap methods for forward gene cloning are in high demand. Indeed the current quest is to find methods that enhance the speed and reduce the costs of forward gene isolation, and in particular to extend the range of species in which gene cloning can be practiced.

The objective in forward gene cloning is the identification of those genes that are known only from a phenotype and for which no molecular information or sequence information is available. The starting point in forward genetics can be naturally occurring phenotypic variants or artificially induced mutants.

Essentially, two groups of methods are being recognized for forward gene cloning: mapping strategies and tagging strategies. They are complementary, each with its inherent limitations.

In mapping strategies, phenotypes are pinpointed to the smallest-most segment of a chromosome by marker-assisted meiotic recombination mapping. Map based cloning is a very laborious procedure and is in particularly employed in a small group of model species.

Theoretically, map based cloning is a universally applicable procedure for any organism that reproduces through a sexual cycle (Peters et. al. (2003) Trends in Plant Science Vol. 8 No. 10 pp 484-491). In practice, however, there are fundamental biological constraints. Most importantly, it depends critically on a good frequency of meiotic recombination in the area of the gene of interest. Secondly, it becomes increasingly difficult in poorly characterized large genomes, for example in plants like lettuce, pepper, onion, and many others, where repetitive DNA hinders the unequivocal mapping of DNA markers.

In tagging strategies, well-characterized biological mutagens (transposons or T-DNA insertions) have been used as effective mutagens and as a tool to clone tagged genes. Insertion of a transposable element into a gene can lead to loss- or gain-of-function, changes in expression pattern, or can have no effect on gene function at all, depending on whether the insertion took place in coding or non-coding regions of the gene. Genes responsible for any newly arising phenotype are retrieved by cloning the insertion sequences along with flanking pieces of genomic DNA.

Tagging with the help of such transposable elements is theoretically a preferable approach to clone genes because it is fast, independent of meiotic recombination, and requires no previous genomic resources such as genetic maps or extensive sequence information (See Maes et al. Trends Plant Sci. 1999 Mar;4(3):90-96.)

The tagging approach has been successful in a handful of model organisms, in which natural gene tagging (transposon) systems were known, or where large numbers of individuals could be transformed with randomly integrating DNA (T-DNA) (see for example Settles et. al, BMC Genomics 2007, 8:116, using maize). Outside of this small group of species, tagging cannot or hardly be used either due to the absence of known insertion elements, or due to logistic constraints on population size. Logistically, tagging requires populations of many thousands of individuals in order to find one or a few specific mutants, as the number of insertion sequences per genome is low (1-200).

It is one of the goals of the present invention to provide for a new method, and the use thereof, that allows for the efficient identification of nucleic acids that are involved in the manifestation of a particular phenotype. The method should be employable in any (plant) species that can be artificially crossed and manipulated, without the need of extensive sequence knowledge of the species. Other goals of the present invention will become apparent from the description of the invention, the embodiments and the claims.

### DEFINITIONS

In the following description and examples several terms are used. In order to provide a clear and consistent understanding of the specification and claims, including the scope to be given to such terms, the following definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

Allele: One of at least two alternative forms of a gene that can have the same place on homologous chromosomes and are responsible for alternative traits. A non-limitative example is a gene for blossom colour in a flower—a single gene might control the colour of the petals, but there may be several different versions or alleles of the gene. One version might result in red petals, while another might result in white petals. The resulting colour of an individual flower will depend on which two alleles it possesses for the gene and how the two interact.

Character: Relates to a phenotypical quality of an organism. A character can manifest itself in different traits. For example, the plant can be a plant, having flower colour as a character, and the red or white flowers being the traits A and B of the character. Within the current invention, the character (or trait) can be any, as long as members of the organism having a first trait of the character can be phenotypically distinguished from members of the organism having a second trait of the character. This is not limited to only differences that can be directly observed by inspection of an organism, but also includes characters/traits that can become apparent upon further analysis of the organism, for example upon analysis of the resistance to certain circumstances, or upon analysis of the presence of particular metabolites in such organism.

“Expression of a gene” or “expressed nucleic acid”: the process wherein a DNA region, which is operably linked to appropriate regulatory regions, particularly a promoter, is transcribed into an RNA, which is biologically active, i.e. which is capable of being translated into a biologically active protein or peptide or which is active itself.

Gene: a DNA sequence comprising a region (transcribed region), which is transcribed into an RNA molecule (e.g. an mRNA) in a cell, operably linked to suitable transcription regulatory regions (e.g. a promoter). A gene may thus comprise several operably linked sequences, such as a promoter, a 5′ non-translated leader sequence (also referred to as 5′UTR, which corresponds to the transcribed mRNA sequence upstream of the translation start codon) comprising e.g. sequences involved in translation initiation, a (protein) coding region (cDNA or genomic DNA) and a 3 ′non-translated sequence (also referred to as 3′ untranslated region, or 3′UTR) comprising e.g. transcription termination sites and polyadenylation site.

Isogenic: Genetically identical. Individual cells within an isogenic population are typically the progeny of a single ancestor, having equal genetic make-up. Within the current invention, “isogenic” is to be construed that at the level of cDNA the individual members are 100% identical except for any point mutation that might arise as a consequence of natural variation, or, in a preferred embodiment of the invention, from a mutagenic treatment.

Mutagenesis or mutagenic treatment: The terms relate herein to a treatment leading to the introduction of changes in nucleic acid, genes, or genomes, for example leads to the introduction of point mutations and/or insertion or deletion of up to 10 consecutive nucleotides.

Nucleic acid: a nucleic acid according to the present invention may include any polymer or oligomer of pyrimidine and purine bases, preferably cytosine, thymine, and uracil, and adenine and guanine, respectively (See Albert L. Lehninger, Principles of Biochemistry, at 793-800 (Worth Pub. 1982) which is herein incorporated by reference in its entirety for all purposes). The nucleic acids may be DNA, including cDNA, or RNA, or a mixture thereof, and may exist permanently or transitionally in single-stranded or double-stranded form, including homoduplex, heteroduplex, and hybrid states.

Sequencing: The term sequencing refers to determining the order of nucleotides (base sequences) in a nucleic acid sample, e.g. DNA or RNA.

Trait: In biology, a trait relates to any phenotypical distinctive character of an individual member of an organism in comparison to (any) other individual member of the same organism. Within the context of the current invention the trait can be inherited, i.e. be passed along to next generations of the organism by means of the genetic information in the organism.

“Trait of the same character” or “trait of said character”: anyone of a group of at least two traits that exist (or became apparent) for a character. For example, in case of the character “colour of the flower”, phenotypical manifestations might comprise blue, red, white, and so on. In the above example blue, red and white are all different traits of the same character.

### DETAILED DESCRIPTION OF THE INVENTION

The above mentioned goals are surprisingly solved by providing for a method as described in the accompanying claims.

More specifically, there is provided a method for identification, and optional isolation, of an expressed nucleic acid sequence that is associated with a character of an organism, characterized in that the method comprises the following steps:
a. Providing at least two members of said organism having a trait A of said character and at least two members of said organism having a trait B of said character, and wherein trait A and trait B are different, and wherein said members having trait A or B are both derived from isogenic members of said organism;
b. Obtaining total cDNA from each of the members of step a) having trait A and from each of the members of step a) having trait B;
c. Determining sequences of each of the individual cDNA's obtained from the members having trait A and from the members having trait B;
d. Determining single nucleotide polymorphism frequencies in each individual cDNA of members having trait A by comparison to corresponding cDNA of members having trait B;
e. Identifying cDNA from the members having trait A with an increased single nucleotide polymorphism frequency by comparing the single nucleotide polymorphism frequency of the cDNA of each of the members having trait A with the cDNA of each of the members having trait B; and
f. Identifying the expressed nucleic acid sequence from which the cDNA of step (e) is derived and, optionally, cloning the gene comprising the expressed nucleic acid sequence.

The current invention is based on the realization that the above mentioned problems with current forward cloning strategies can be solved by a method that is conceptually similar to tagging with biological mutagens, but yet overcomes the limitations thereof, by using non-biological mutagens that can be applied to any organism, and which produces many thousands of well-detectable mutations per genome to lift logistical population constraints as much as possible, in combination with sequencing of the whole transcriptomes (cDNA) obtained from a mutant pool showing a desired phenotype, and based thereupon identifying a gene which carries a non-neutral single nucleotide polymorphism in each of the members of said mutant pool. In addition, the mutant pool can be obtained from natural variation.

As in gene tagging with insertion mutagens, it was realized that genes in any organism can be tagged (and manifest a distinct phenotype) with chemically induced mutations like point mutations. However, a major problem lies in how to detect such small and random mutations (tags) in the whole genome, as such mutation does not qualify for any form of specific PCR amplification. Moreover, chemically induced mutations are randomly introduced in the genome with high frequency. Consequently, many genes might concurrently comprise mutations in comparison to the genes of the original species, further complicating the correct identification of the gene responsible for an observed phenotype.

The inventors have realized that the solution comes from creating a series of mutant alleles at the locus to be cloned, pooling them into a “mutant pool” and then re-sequencing all cDNA from this pool multiple times. When compared to all cDNA sequence from a pool of non-mutant siblings (the “wild type pool”) one cDNA in the mutant pool will show a highly increased mutation frequency. This is extremely likely a gene underlying the mutant phenotype.

In the method according to the invention, pools are made from an (induced) allelic series in an otherwise isogenic genetic background. The method according to the invention (Bulked Mutant Analysis, BMA) detects linked SNPs that are located within the gene to be cloned.

The method is applicable to all species that can be artificially hybridized and mutagenised, including such notoriously cumbersome crops as peppers and onions, is independent of the presence of a genetic map, and will work in genomes of any size and complexity.

In more detail, in the method according to the invention at least two individual members of an organism having a particular phenotype (or having a particular trait A) are compared with at least two individual members of said organism not having said phenotype (but having a trait B with respect to the same character). The skilled person will understand that the current method is not limited to only comparing members having a trait A and members having a trait B, but might also include members having a trait C, D, E, . . . etc, of the same character.

For the invention it is required that the members of the organism having trait A or trait B are both derived from isogenic members of said organism, in other words from organisms having the same genetic background (for example are derived from the same inbred strain). The individual members having trait A or trait B are isogenic, i.e. have the same genetic background, except for changes that were introduced into the genetic material due to natural variation or, in a preferred embodiment of the invention, due to a mutagenic treatment. It is in these differences in the genetic material between the members having trait A and the members having trait B that the observed phenotype is comprised. The members having trait A must express at least one nucleic acid that is different from the nucleic acids expressed by the members of the organism having trait B, and which nucleic acid is associated with the character of the organism, of which trait A and trait B are phenotypical forms.

In order to detect such tagged (by, for example, a point mutation) nucleic acid, in the method of the current invention, the total transcriptomes of the members having trait A are compared with the total transcriptomes of the members having trait B, i.e. the complete sequence of all expressed genes in a selected tissue are compared.

For this total cDNA is obtained from both the members having trait A and from the members having trait B. Preparation of total cDNA from each of the pool of members of the organism having trait A or of the organisms having trait B can be done by any suitable method known to the skilled person. Many commercially available kits for cDNA synthesis can be purchased, such as e.g. from ABgene, Ambion, Applied Biosystems, BioChain, Bio-Rad, Clontech, GE Healthcare, GeneChoice, Invitrogen, Novagen, Qiagen, Roche Applied Science, Stratagene, and the like. Such methods are e.g. described in Sambrook et al . (Sambrook, J., Fritsch, E. F., and Maniatis, T., in Molecular Cloning: A Laboratory Manual. Cold Spring Harbor Laboratory Press, NY, Vol. 1, 2, 3 (1989)).

It is required that, for the method of the current invention, that total cDNA of at least two members having trait A and at least two members having trait B, are obtained. More preferably, at least 3, 4, 5, 6 or 7 members of the organisms, and each having a first trait A are provided in the method according to the invention.

As explained above, the current invention is based on the realization that a genetic difference that is associated with a particular character (or trait) can be identified by comparing the sequences of the whole transcriptomes of organisms that can be distinguished from each other by having phenotypical distinct traits. However, since in a preferred embodiment the members having a trait A are generated by random (chemical) mutagenesis treatment of members having a trait B, the genetic material of such treated organisms will comprise many and random mutations, and of which most of them will not be associated to the observed traits. Comparing the total cDNA of one member having trait A to the total cDNA of one member having trait B will thus not allow for the identification of the nucleic acid associated with the observed phenotype. It was however realized by the current inventor that when at least two members, each having a first trait A are compared with at least two members having trait B, it now becomes possible to identify the nucleic acid that is responsible (or associated) for the character (or trait):

The members having trait A and trait B are both derived from the same isogenic source. As a consequence of a mutation treatment of the isogenic source, alterations are randomly introduced in the genetic material. The alterations induced in a first member will be different from the alterations induced in a second member. However, in case both said first member and said second member, as a consequence of the mutagenic treatment, now express a phenotype, i.e. have a trait A, it is extremely likely that in both members the same cDNA will display a single nucleotide polymorphism (SNP) in comparison to the corresponding cDNA obtained from members having trait B (such SNP does not necessarily has to be localized at the same position within such cDNA). Said cDNA can now be identified as a cDNA derived from an expressed nucleic acid that is associated with a particular trait or character of the organism under study, as the change that a non-associated cDNA would, in both members having trait A, display such SNPs is near zero.

In other words, the current invention is based on the realization that in the sequences of all cDNA from a “mutant pool” ,of e.g. 5 allelic mutations in one gene (trait A), there will be just one individual cDNA that is consistently showing sequence changes in comparison to the cDNA of the members having trait B.

In order the compare the total cDNA of members having trait A with the total cDNA of members having trait B, the total cDNA needs to be sequenced. Sequencing of cDNA can be done by any suitable method known to the skilled person. However, in particular such methods as described by Margulies M. et al. (http://www.454.com, Genome sequencing in microfabricated high-density picolitre reactors. Nature 437:376-80, 2005] are highly preferably, allowing for rapid and efficient sequencing of the whole transcriptome (all cDNA's). For example, and in a preferred embodiment, the nucleotide sequences of the obtained cDNA fragments are determined by high- throughput sequencing methods, for example like those disclosed in WO 03/004690, WO 03/054142, WO 2004/069849, WO 2004/070005, WO 2004/070007, and WO 2005/003375, by Seo et al. (2004) Proc. Natl. Acad. Sci. USA 101:5488-93, and technologies of Helicos, Solexa, US Genomics, etcetera, which are herein incorporated by reference.

In a next step in the method according to the invention, the obtained sequences of the total cDNA of the members having trait A are compared to the total cDNA of the members having trait B in order to establish single nucleotide polymorphism frequencies in the cDNA's. This determination can be done by any suitable method known to the skilled person, and for example as set-out in the accompanying example.

In short, for example, alignment of the nucleotide sequences of cDNA fragments may be used to collect nucleotide sequences derived from the same transcribed gene, and to compare these nucleotide sequences. Whether nucleotide sequences are derived from a same transcribed gene can be established based on homology between the sequences. For the purposes of this invention, it is assumed that nucleotide sequences are derived from a same transcribed gene when they are at least 95, 96, 97, 98, 99, 100 per cent homologous over a length of at least 30, preferably at least 50, more preferably at least 90, yet more preferably at least 100, 150, 200 nucleotides. The method may be aided by statistical interpretations to demonstrate statistically different frequencies.

Based on the obtained data, a cDNA in the total cDNA of the members having trait A having an increased SNP frequency can be identified. In the method according to the invention, among all the cDNA sequenced in the members having trait A, at least one specific cDNA will carry a genetically non-neutral single nucleotide polymorphism (SNP) in each of members having trait A. In other words, this specific cDNA will comprise a SNP in each of members having trait A (not necessarily on the same localization in the corresponding cDNA's). Such cDNA can subsequently be used to identify the expressed nucleic acid sequence and clone the corresponding gene by methods known to the skilled person.

In a preferred embodiment, there is provided for a method according to the invention wherein the members of said organism having trait A were obtained by mutagenesis of members of said organism having said trait B, and wherein said members having trait B where isogenic before said mutagenesis treatment.

In an embodiment of the method according to the invention, the at least two members having a trait A of a distinct character can be the consequence of natural variation, i.e. due to natural or spontaneous changes in a nucleic acid is said organism previously manifesting trait B. Such mutations in the genetic information are unintentionally, but reveal that the organism carries genetic information responsible for the observed change in phenotype (for example, from trait B to trait A).

Preferably however, the method according to the invention does not depend on such unintentional and uncontrollable variation in the genetic information, but instead depends on the mutants having a particular trait being the result of deliberate mutagenesis. The skilled person understands how he can mutate the genetic information of any organism, for example by the use of known mutagens. Due to the use of such mutagens, mutations can randomly occur in the genome of members of the organisms. In other words, nucleic acids, like genes, can be tagged (by comparison to non-mutated nucleic acids) with (chemically, biologically or by means of radiation) induced mutations (e.g. point mutations, e.g. by ethylmethanesulfonate).

Examples of “mutagenesis by irradiation” include X-rays, y-rays, UV light, or ionizing particles. Examples of “biological mutagenesis” include, for example, such methods as described in W00150847, for example using recombinases like recA. Examples of “chemical mutagens” that can be used in the method according to the invention include the application of specific chemicals such as ethylmethanesulfonate (EMS), diethyl sulphate (DES), N-nitroso-N-ethylurea (ENU), diepoxybutane, 2-aminopurine, 5-bromouracil, ethidiumbromide, nitrous acid, nitrosoguanidine, hydroxylamine, sodium azide, or formaldehyde.

Preferably, the method of mutagenesis induces point mutations in the genome, or insertion, substitution or deletion of up to 10 consecutive nucleotides.

The skilled person will understand that within the context of this embodiment the members having trait A and the members having trait B are isogenic (as they are both derived from the same genetic background), except for the mutations introduced by said mutagenesis treatment.

In contrast to classical mapping and tagging strategies with insert mutations however, such small and random mutations are difficult to detect as they do not qualify for any form of specific PCR amplification. However, there are several good reasons to use the above strategy: the mutagenesis can be applied to essentially any species of interest, the spectrum of induced mutants is broader than with tagging approaches, mutagenesis is usually more efficient and second-site mutations are easier to obtain.

Although a wide variety of suitable method of mutagenesis can be employed, preferably, the mutagens are not a biological mutagens selected from the group consisting of transposon inserts or T-DNA inserts. Preferably, the used method of mutagenesis introduces point mutations in the genetic material.

In another embodiment there is provided for a method according to the invention and wherein the organism is a plant, preferably a crop plant selected from the group consisting of tomato, pepper, aubergine, lettuce, carrot, onion, leek, chicory, radish, parsley, spinach, melon, cucumber.

The organism that can be subject to the current invention can be any organism, including bacteria, prokaryotes and eukaryotes. However preferably the organism is an eukaryote, in particular a plant, more in particular a crop plant. Preferably, the plant is a plant belonging to the group consisting of important crop plants, including tomato, pepper, aubergine, lettuce, carrot, but the method can be applied to essentially all others plants.

In particular, the current invention allows for the identification and cloning of nucleic acids, like genes, from crop plants in which mapping and tagging techniques available in the art turn out to be impossible or cumbersome.

In a further embodiment there is provided for a method according to the invention wherein the organism is a plant and wherein prior to step a) a F1 population that is heterozygous for an allele encoding for a trait A and a second allele encoding for a trait B is created, and wherein said F1 population is subjected to mutagenesis and wherein said F1 population is after said mutagenesis divided in members having trait A and in members having trait B.

Preferably, the allelic series can for example be constructed by a standard genetic approach for selecting new alleles at a known locus. This approach consists of creating a F1 population that is heterozygous for one mutant reference allele (which produces the previously known phenotype of the locus) and one wild type allele. Mutagenesis of the F1 will uncover the mutant phenotype by knock out of the wild type allele. Such mutant F1 plants will appear at a certain frequency in the otherwise wild type F1 population. The number of different genes (loci) to be treated this way in one F1 population can be increased at will, by combining different mutant loci in one multiple heterozygous genotype.

The nucleic acid that will be identified by the method according to the invention can subsequently be isolated, cloned (gene), or introduced in a host cell, or used in for example, plant breeding programs.

In summary, the current invention relates to a new strategy for identification, and optional isolation, of a nucleic acid sequence that is expressed in an organism and that is related to a particular phenotype (trait of a character) of said organism. With the method of the current invention is has become possible to, in contrast to known methods in the art, efficiently identify, isolate or clone genes in, for example, organism like (crop) plants for which no or only limited information with respect to the genome is available. In addition, it is now possible to take advantage of the use of chemicals inducing point mutations as a versatile, broadly applicable method for creating mutagenised populations, for example as much higher mutation frequencies than by using T-DNA or transposon system are generated, thereby reducing the amount of organisms, for example plants, needed in the method. Moreover, the method does not rely on the use or presence of markers in the genome, as it directly detects changes in a gene of interest./esp

### FIGURES

FIG. 1 is a schematic overview of an embodiment of the method according to the invention. In the overview, and applicable to the invention disclosed herein, it is shown that a isogenic population is treated with a mutagens. As a consequence, two traits A (dashed circles) and B (continuous circles) of a particular distinct character became apparent. The two traits A and B are thus derived from the same isogenic population, and differ in this embodiment only with respect to the mutations induced by the mutagenesis treatment (a skilled person will understand that said treatment might have been performed on all isogenic members of the organism or, preferably, to only a fraction of the members of said organism). In order to identify an expressed nucleic acid that is responsible or associated with the observed trait A, at least two members having trait A are pooled, total cDNA is obtained, sequenced and compared with total cDNA of at least two members having treat B (or, compared to total cDNA previously obtained from members having trait B, for example from the isogenic members before the mutagenesis treatment). Due to the pooling of at least two members having trait A, the chance that random mutations were introduced in the same cDNA in all members of the mutant pool, and that such cDNA is not involved in the observed phenotype is practical zero. If, in a next stage, the total cDNA of the members having trait A and having trait B are compared, such cDNA that carries mutations in all members in the mutant pool having trait A can be identified as being a nucleic acid involved in the observed phenotype/trait A. In the overview this is represented by showing the comparison of total cDNA of trait A with total cDNA of trait B for two individual cDNA's: cDNA1 and cDNA2. As can be witnessed, cDNA1 from trait A and trait B, are, except for one member of trait A, identical. In contrast all cDNA2 of trait A carry a mutation (shown by a star) in comparison to the cDNA2 of trait B. cDNA2 is consequently identified as a nucleic acid involved/associated in the appearance of trait A, and the gene can be cloned.

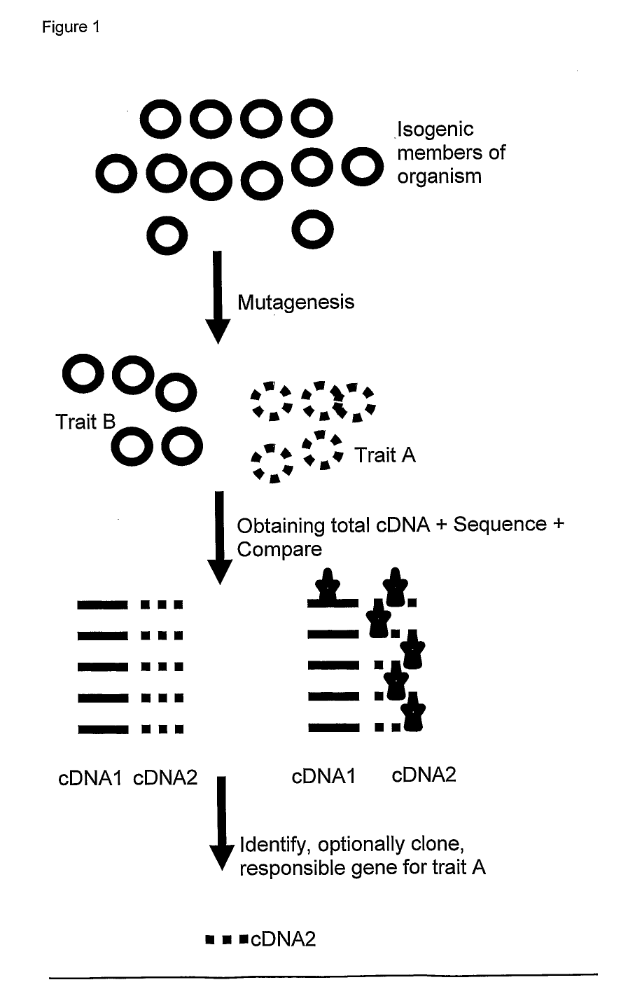

### EXAMPLE

Generation of mutants in a single flower colour gene.

With the below example, the BMA method can be demonstrated. The method can be demonstrated by the de novo identification of a previously known flower colour gene (R7) of Petunia hybrida. A magenta flowering F1 hybrid Petunia can be produced from a cross between the inbred lines W5 (rt:::dTph3; Stuurman and Kuhlemeier. 2005 and M1 (RT, Snowden KC and Napoli CA (1998) PsI: a novel Spm-like transposable element from Petunia hybrida. Plant J. 14:43-54), which results in a magenta-flowering heterozygous genotype carrying a recessive allele at the gene RT, which codes for UDP rhamnose: anthocyanin-3-glucoside rhamnosyl transferase. In the genetic background used, null mutants of RT will produce red flowers, as a consequence of the accumulation of cyanidin-3-glucoside (Kroon et al. 1994, Brugliera et al. 1994). Upon mutagenic treatment of F1 hybrid seeds with ethylmethanesulfonate (EMS), some of the resulting plants will carry novel mutations in the wild type allele of RT resulting in loss of heterozygosity and expression of a red corolla colour.

A population of about 3600 F1 plants can be grown from EMS treated seed, and flower colour can be recorded visually on all individual plants. From the grown plants, five red-flowering mutants can be selected. Because red colour is recessive, the expression of this colour in the primary mutagenised population indicates that the red-flowering mutants carried EMS induced point mutations in the wild type copy of the RT gene.

High throughput transcriptome sequencing identifies the RT gene correctly

To de novo identify the gene underlying the red colour in the 5 newly identified mutant alleles, the full transcriptome of stage 2 corolla limbs can be sequenced, which is the developmental time point at which anthocyanin pigmentation becomes visible. At the same time full transcriptome of at least two plants from seeds that where non-EMS treated (hereafter referred to as wild type) can be sequenced for comparison. Total RNA of each of the 5 mutants and wild type can be converted into ds-cDNA, and cDNA from each individual mutant plant is than separately processed for sequencing on a GS-FLX Titanium sequencer (454 Life Sciences). Processing can involve a bar-coded 454-sequencing adapter, with each of the 5 mutants and wild type carrying a distinct bar-code (see for example WO2007073165, WO2007037678 or WO2007073165, wherein the distinct bar-code is described as a tag or identifier). This bar-code can be a 5 bp sequence, added to the 3′ end of the 454-adapter, which is read along with the cDNA. This will generate a unique tag at the beginning of each sequence read, the exact 5 bp sequence of which indicates from which of the 5 mutants or wild type it was derived. All samples can subsequently be pooled, and sequenced with 3 runs of GS-FLX Titanium sequencing. A total of 3 million reads of average 400 bp length can be obtained.

All sequence data can be assembled into a non-redundant set of contigs using CAP3 assembly software (Huang, X. and Madan, A. (1999) CAP3: A DNA Sequence Assembly Program. Genome Research, 9: 868-877) using a 98% identity setting for overlap matching. About 20 000 contigs of average 1.5 kb size can be obtained, representing the minimum number of unigenes for the corolla transcriptome at this depth of sequencing.

The dataset is than analyzed for the occurrence of SNPs in the mutants, and in comparison to the wild type, using techniques known to the skilled person (Savage et al. 2005. SNPserver, al real timeSNP discovery tool Nucl. Acids Res (33) W493-W495). When used in a BLAST analysis, this unigene will show to be identical to UDP rhamnose: anthocyanin-3-glucoside rhamnosyl transferase from Petunia hybrida. This is the same at the RT gene.

The BMA procedure allows the identification of a single gene that causes a mutant phenotype within a background of at least 21000 other genes, provided that experimental conditions allow the generation of an allelic series (in our case 5 distinct mutants in the same gene) and sufficient sequencing power to detect SNPs in pools of whole-tissue cDNA.

1. A method for identification, and optional isolation, of an expressed nucleic acid sequence that is associated with a character of an organism, comprising the steps of:
a. providing at least two members of said organism having a trait A of said character and at least two members of said organism having a trait B of said character, and wherein trait A and trait B are different, and wherein said members having trait A or B are both derived from isogenic members of said organism;
b. Obtaining total cDNA from each of the members of step a) having trait A and from each of the members of step a) having trait B;
c. Determining sequences of each of the individual cDNA's obtained from the members having trait A and from the members having trait B;
d. Determining single nucleotide polymorphism frequencies in each individual cDNA of members having trait A by comparison to corresponding cDNA of members having trait B;
e. Identifying cDNA from the members having trait A with an increased single nucleotide polymorphism frequency by comparing the single nucleotide polymorphism frequency of the cDNA of each of the members having trait A with the cDNA of each of the members having trait B; and
f. Identifying the expressed nucleic acid sequence from which the cDNA of step (e) is derived and, optionally, cloning the gene comprising the expressed nucleic acid sequence.

2. The method according to claim 1 wherein the members of said organism having trait A were obtained by mutagenesis of members of said organism having said trait B, and wherein said members having trait B where isogenic before said mutagenesis treatment.

3. The method according to claim 2 wherein the method of mutagenesis induces point mutations.

4. The method according to claim 3 wherein mutagenesis is performed by use of a non-biological mutagens.

5. (canceled)

6. (canceled)

7. The method according to claim 4 wherein the organism is a plant, preferably a crops plant selected from the group consisting of tomato, pepper, aubergine, lettuce, carrot, onion, leek, chicory, radish, parsley, spinach, melon, cucumber.

8. The method according to claim 7 wherein the organism is a plant and wherein prior to step (a) a F1 population that is heterozygous for an allele encoding for a trait A and a second allele encoding for a trait B is created, and wherein said F1 population is subjected to mutagenesis and wherein said F1 population is after said mutagenesis divided in members having trait A and in members having trait B.

9. The method according to claim 8 wherein the gene of step (f) is introduced in an organism, and/or is used to create a transgenic organism, and/or is mutated, and/or is used in plant breeding.
